(19) United States
(12) Patent Application Publication
(10) Pub. No.: US 2014/0257089 A1
(43) Pub. Date: Sep. 11, 2014

(54) MEANS AND METHODS FOR THE PREVENTION OF RETAINED SURGICAL ITEMS AND GOSSYPIBOMA

(71) Applicant: Aspect Imaging Ltd., Shoham, IL (US)

(72) Inventor: Uri RAPOPORT, Moshav Ben Shemen (IL)

(21) Appl. No.: 13/939,625

(22) Filed: Jul. 11, 2013

Related U.S. Application Data
(60) Provisional application No. 61/775,604, filed on Mar. 10, 2013.

(30) Foreign Application Priority Data
Mar. 5, 2013 (DE) 20 2013 100 949.0

(51) Int. Cl. A61B 5/06

(52) U.S. Cl. CPC A61B 5/062
USPC 600/424

(57) ABSTRACT

A standard of care protocol for preventing gossypiboma during surgery. The protocol including: selecting one or more surgical items from a group consisting of: non-metallic surgical items marked with detectable metal indicia; metallic surgical items; metallic surgical items marked with the detectable metal indicia; providing one or more metal detectors in connection with the selected at least one of the surgical items; and identifying, using at least one metal detector, detectable metal items in the patient.

### FIELD OF THE INVENTION

The present invention pertains to safety during surgical and imaging procedures and more particularly to a standard of care for prevention of gossypiboma during such procedures.

### BACKGROUND OF THE INVENTION

Retained surgical items such as surgical instruments and sponges are seldom reported due to medicolegal implications.

Patent application WO2010008846 is directed towards an automated detection and tracking system for recognizing and identifying sharp objects from intra-operative surgical image data. Patent application WO2008147573 is directed towards an operating room surgical items tracking system for hospitals to identify multiple specific items. The prior art systems are time consuming and not effective in situ. In addition, they are not applicable to search for left surgical items in retrospect in a non-laborious way.

There thus remains a long felt need for standard of care methods in the hospital environment adapted to (1) screen subjects for metals before entering an MRI room and (2) screen subjects undergoing surgery or minimally invasive surgeries for preventing gossypiboma.

### SUMMARY OF THE INVENTION

The present invention provides a standard of care protocol for preventing gossypiboma during open surgery (100), comprising steps of: (a) providing a metal detector (101) comprising: (i) at least one magnetic field sensor for identifying detectable metals, (ii) at least one reporting mechanism adapted to signal data concerned with detection of the detectable metals; and (iii) a CPU for processing the data; (b) identifying detectable metal items in a patient (103); wherein the identifying step comprises steps of processing the data to establish a baseline threshold signal (102) characteristic of pre-surgery detectable metals in a patient; and then identifying signals exceeding determine baseline threshold in a time resolved manner during and after the open surgery procedure, thereby facilitating the prevention of gossypiboma.

It is another object of the current invention to disclose standard of care protocol for preventing gossypiboma during surgery, characterized by steps of a. selecting one or more surgical items from a group consisting of: non-metallic surgical items marked with detectable metal indicia (210); metallic surgical items (220); metallic surgical items marked with said detectable metal indicia (230) and a combination thereof; b. providing one or more metal detectors in connection with at least one of said surgical items; and c. identifying by mean of said at least one metal detector detectable metal items in said patient.

It is another object of the current invention to disclose the standard of care protocol as defined in any of the above wherein the detectable metal items include non-metallic surgical items marked with detectable metal indicia.

It is another object of the current invention to disclose the standard of care protocol as defined in any of the above wherein the non-metallic surgical items are selected from a group consisting of: pads, sponges, stents, tapes, gauzes, dressings, staples, stitches, threads, sutures, tubes, plastic items, rubber items and any combination thereof.

It is another object of the current invention to disclose the standard of care protocol as defined in any of the above wherein the detectable metal items include metallic surgical items.

It is another object of the current invention to disclose the standard of care protocol as defined in any of the above wherein the metallic surgical items are marked with detectable metal indicia.

It is another object of the current invention to disclose the standard of care protocol as defined in any of the above wherein the metallic surgical items are selected from a group consisting of: graspers, forceps, clamps, occluders, retractors, ribs and other tissue distractors, positioners, stereotactic devices, mechanical cutters scalpels, lancets, drill bits, rasps, trocars, ligasure, harmonic scalpel, dilators and specula, suction tips and tubes, sealing devices, surgical staplers, irrigation and injection needles, tips and tubes, tyndallers, drills, dermatomes; and any combination thereof.

It is another object of the current invention to disclose the standard of care protocol as defined in any of the above wherein the detectable metal indicia are distinct to each surgical item in a manner selected from a group consisting of: (a) type of detectable metal; (b) geometry; (c) amount of metal; and any combination thereof.

It is another object of the current invention to disclose the standard of care protocol as defined in any of the above wherein the detectable metal indicia are interconnected to the surgical item in a way selected from a group consisting of: welding, affixing, embedding, interweaving, coupling, casting, joining, implanting, attaching, linking, screwing, adhering, stamping, forming, bending; and any combination thereof.

It is another object of the current invention to disclose the standard of care protocol as defined in any of the above wherein there is at least one detectable metal indicium in a surgical item.

It is another object of the current invention to disclose the metal detector as defined in any of the above wherein the metal detector is hand-held.

It is another object of the current invention to disclose the standard of care protocol as defined in any of the above wherein the metal detector is embedded within a portable medical device.

It is another object of the current invention to disclose the standard of care protocol as defined in any of the above wherein the metal detector is embedded within a surgical implement proximate to the operating room.

It is another object of the current invention to disclose the standard of care protocol as defined in any of the above wherein the metal detector is additionally comprising: (1) means for evaluating parameters from a group consisting of: (a) type, (b) distance, (c) size, (d) depth, (e) location, and (f) amount of detectable metals and any combination thereof; (2) means for reporting these parameters and any of their combination thereof by producing a signal.

It is another object of the current invention to disclose the standard of care protocol as defined in any of the above wherein the signal is selected from a group of indicators consisting of: (a) audible indicators; (b) visual indicators; and (c) sensible indicators; and any combination thereof.

It is another object of the current invention to disclose the standard of care protocol as defined in any of the above wherein, the signal intensity is characterized as a function of at least one parameter from a group consisting of (a) type; (b) distance; (c) size; (d) depth; (e) location; and (f) amount of detectable metals; and any combination thereof.

It is another object of the current invention to disclose a standard of care protocol for preventing gossypiboma during a minimally invasive surgery (300), comprising steps of: (a) providing a metal detector adapted to identify detectable metal items in a body cavity of a patient (301); (b) at least partially introducing the metal detector into a body cavity of the patient (302); (c) identifying detectable metal items in real-time (303); (d) alerting on the identification of detectable metal items in real-time (4); wherein the metal detector is characterized by: (a) at least one magnetic field sensor for identifying detectable metals embedded within the distal tip of the endoscope; (b) at least one reporting mechanism adapted to signal on the detection of detectable metals embedded within the external parts of the endoscope; thereby facilitating the prevention of gossypiboma.

It is another object of the current invention to disclose the standard of care protocol as defined in any of the above wherein the endoscope refers to any tool selected from a group consisting of: endoscopes, sigmoidoscope; proctoscope; rhinoscope; bronchoscope; otoscope; gynoscope; laparoscope; amnioscope; laryngoscope; esophagoscope; bronchoscope; epiduroscope; and any combination thereof.

It is another object of the current invention to disclose the standard of care protocol as defined in any of the above wherein minimally invasive surgery refers to any procedure selected from a group consisting of: endoscopy; laparoscopy; NOTE (Natural orifice translumenal endoscopic surgery); NOS (Natural Orifice Surgery); and any combination thereof.

It is another object of the current invention to disclose the standard of care protocol as defined in any of the above wherein the detectable metal items include non-metallic surgical items marked with detectable metal indicia.

It is another object of the current invention to disclose the standard of care protocol as defined in any of the above wherein the non-metallic surgical items are selected from a group consisting of: pads, sponges, tapes, gauzes, dressings, stitches, threads, sutures, tubes, plastic items, rubber items and any combination thereof.

It is another object of the current invention to disclose the standard of care protocol as defined in any of the above wherein the detectable metal items include metallic surgical items.

It is another object of the current invention to disclose the standard of care protocol as defined in any of the above wherein the metallic surgical items are marked with detectable metal indicia.

It is another object of the current invention to disclose the standard of care protocol as defined in any of the above wherein the metallic surgical items are selected from a group consisting of: graspers, forceps, clamps, occluders, retractors, ribs and other tissue distractors, positioners, stereotactic devices, mechanical cutters scalpels, lancets, drill bits, rasps, trocars, ligasure, harmonic scalpel, dilators and specula, suction tips and tubes, sealing devices, surgical staplers, irrigation and injection needles, tips and tubes, tyndallers, drills, dermatomes; and any combination thereof.

It is another object of the current invention to disclose the standard of care protocol as defined in any of the above wherein the detectable metal indicia is distinct to each surgical item in a manner selected from a group consisting of: (a) type of detectable metal; (b) geometry; (c) amount of metal; and any combination thereof.

It is another object of the current invention to disclose the standard of care protocol as defined in any of the above wherein the detectable metal indicia are interconnected to the surgical item in a way selected from a group consisting of: welding, affixing, embedding, interweaving, coupling, joining, implanting, attaching, linking, screwing, adhering, stamping, forming; and any combination thereof.

It is another object of the current invention to disclose the standard of care protocol as defined in any of the above wherein there is at least one detectable metal indicium in a surgical item.

It is another object of the current invention to disclose the standard of care protocol as defined in any of the above wherein the endoscope is characterized by having: (a) at least one rigid or flexible tube; (b) at least one light delivery system; (c) at least one lens system; (d) at least one eyepiece and (e) at least one additional channel adapted to introduce least one medical instrument or manipulator.

It is another object of the current invention to disclose a standard of care protocol for preventing gossypiboma while using a toolbox system for interchanging metal comprising surgical items within a body cavity during a minimally invasive surgery (500), comprising steps of: (a) Providing a toolbox system (501) characterized by: (i) at least one anchoring unit adapted to be at least partially reversibly anchored to an internal wall within a body cavity; (ii) at least one interchanging metal comprising surgical item adapted to be reversibly attached to the anchoring unit; (iii) a metal detector embedded within the anchoring unit adapted to detect changes in the amount of interchanging metal comprising surgical items attached to it and alert on changes; (b) introducing the toolbox system into a body cavity (502); (c) reversibly anchoring the toolbox system to the internal wall within the body cavity (503) and (d) reversibly attaching at least one interchanging metal comprising surgical items to the anchoring unit; thereby providing at least one interchanging metal comprising surgical item within the body cavity (504); wherein the metal detector is adapted to alert whenever at least one of the interchanging metal comprising surgical items is detached from the anchoring unit, thereby facilitating the prevention of gossypiboma.

It is another object of the current invention to disclose the standard of care protocol as defined in any of the above wherein the metal detector comprises: (1) a magnetic field sensor adapted to detect changes in the magnetic field in the proximity of the anchoring unit as a result of attaching/detaching the interchanging metal comprising surgical items; (2) a reporting mechanism adapted to alert upon changes in the magnetic field.

It is another object of the current invention to disclose the standard of care protocol as defined in any of the above wherein additionally comprising a step of calibrating the metal detector according to the number of interchanging metal comprising surgical items attached to it.

It is another object of the current invention to disclose the standard of care protocol as defined in any of the above wherein the interchanging metal comprising surgical items include interchanging non-metallic surgical items marked with detectable metal indicia.

It is another object of the current invention to disclose the standard of care protocol as defined in any of the above wherein the non-metallic surgical items are selected from a group consisting of: pads, sponges, tapes, gauzes, dressings, stitches, threads, sutures, tubes, plastic items, rubber items and any combination thereof.

It is another object of the current invention to disclose the standard of care protocol as defined in any of the above wherein the interchanging metal comprising surgical items are selected from a group consisting of: graspers, forceps, clamps, occluders, retractors, ribs and other tissue distractors, positioners, stereotactic devices, mechanical cutters scalpels, lancets, drill bits, rasps, trocars, ligasure, harmonic scalpel, dilators and specula, suction tips and tubes, sealing devices, surgical staplers, irrigation and injection needles, tips and tubes, tyndallers, drills, dermatomes; and any combination thereof.

It is another object of the current invention to disclose the standard of care protocol as defined in any of the above wherein the interchanging metal comprising surgical items are marked with detectable metal indicia.

It is another object of the current invention to disclose the standard of care protocol as defined in any of the above wherein the reporting mechanism is selected from a group consisting of: (a) visual signal; and (b) audible signal; (c) sensible signal and any combination thereof.

It is another object of the current invention to disclose the standard of care protocol as defined in any of the above wherein the visual signal appears in a location selected from a group consisting of: (a) the surface of the toolbox system; (b) the external parts of the invasive surgery apparatus; (c) the internal parts of the invasive surgery apparatus; and any combination thereof.

It is another object of the current invention to disclose the standard of care protocol as defined in any of the above wherein the visual signal is at least one light.

It is another object of the current invention to disclose the standard of care protocol as defined in any of the above wherein the visual light is adapted to change in response to the amount of interchanging metal comprising surgical items attached to the anchoring unit of the toolbox system in a manner selected from a group consisting of: (a) color; (b) intensity; (c) amount; and any combination thereof.

It is another object of the current invention to disclose the standard of care protocol as defined in any of the above wherein the reporting mechanism is interconnected to objects selected from a group consisting of: (a) entrance door; (b) wall; (c) tool tray; (d) personnel uniform; (e) items carried by personnel; and any combination thereof.

It is another object of the current invention to disclose a a standard of care protocol for increasing MRI safety by screening subjects for detectable metals prior their entrance to the MRI room utilizing a hand-held metal detector (600), comprising steps of: (a) providing the metal detector (601); (b) introducing the metal detector to a subject (602); (c) identifying the presence of detectable metals by the magnetic field sensor; (d) alerting on the detection of detectable metals by at least one action from a group consisting of: (1) locking the entrance door to the MRI room (702); (2) shutting down the MRI apparatus (705); (3) shutting down the protective cover of the MRI bore (703); (4) producing an audible or visual alert; and any combination thereof; wherein the metal detector is embedded within a portable medical device.

It is another object of the current invention to disclose the standard of care protocol as defined in any of the above wherein the metal detector is characterized by: (a) at least one magnetic field sensor for identifying detectable metals; (b) at least one reporting mechanism adapted to alert upon the detection by the magnetic field sensor.

It is another object of the current invention to disclose the standard of care protocol as defined in any of the above wherein the metal detector is comprised of at least one magnetic field sensor.

It is another object of the current invention to disclose the standard of care protocol as defined in any of the above wherein the magnetic field sensors are positioned at an angle A relative to each other, where A is in the range of about 0 degrees to about 360 degrees.

It is another object of the current invention to disclose the standard of care protocol as defined in any of the above wherein the metal detector is comprised of three magnetic field sensors adapted to detect magnetic fields in three perpendicular directions.

It is another object of the current invention to disclose the standard of care protocol as defined in any of the above wherein the metal detector additionally comprising: (a) means for evaluating parameters from a group consisting of (i) type; (ii) distance; (iii) size; (iv) depth; (v) location; and (vi) amount of detectable metals; and any combination thereof; (b) means for reporting these parameters and any of their combination thereof.

It is another object of the current invention to disclose the standard of care protocol as defined in any of the above wherein the signal produced by the reporting mechanism is selected from a group of indicators consisting of (a) audible indicators; (b) visual indicators; and (c) sensible indicators; and any combination thereof.

It is another object of the current invention to disclose the standard of care protocol as defined in any of the above wherein the signal intensity is characterized as a function of at least one parameter from a group consisting of (a) type; (b) distance; (c) size; (d) depth; (e) location; and (f) amount of detectable metals; and any combination thereof.

It is another object of the current invention to disclose the standard of care protocol as defined in any of the above wherein at least one additional reporting mechanism is embedded to at least one additional device.

It is another object of the current invention to disclose the standard of care protocol as defined in any of the above wherein an additional reporting mechanism affects another device in a manner selected from a group consisting of: (1) turns on a signal; (2) shuts it down; and (3) locks it; and any combination thereof.

It is another object of the current invention to disclose the standard of care protocol as defined in any of the above wherein the portable medical device is a stethoscope.

It is another object of the current invention to disclose the standard of care protocol as defined in any of the above wherein the stethoscope comprising: (a) at least one chest piece adapted to reversibly contact a subject; (b) at least one ear tip; and (c) tubing interconnecting the chest piece with the ear tips.

It is another object of the current invention to disclose the standard of care protocol as defined in any of the above wherein the magnetic field sensor and the reporting mechanism are embedded within one or more of the stethoscope's parts: (a) chest piece; (b) ear tips; (c) tubing; and any combination thereof.

It is another object of the current invention to disclose the standard of care protocol as defined in any of the above wherein the stethoscope is adapted to alert on detectable metals by either directly or indirectly contacting the stethoscope with a subject.

It is another object of the current invention to disclose a standard of care protocol for increasing MRI safety by screening subjects for detectable metals prior their entrance to an MRI utilizing metal detector gate (50), comprises steps of: (a) providing a metal detector gate (1); (b) positioning the metal detector gate in a location selected from a group consisting of: (i) entrance to MRI room (1); (ii) patient table (2); and (iii) MRI bore (3) (2); (c) passing of the subject through the metal detector gate (3); (d) alerting on the presence of detectable metals; wherein step (d) of alerting is selected from a group consisting of: (1) locking entrance door to MRI room; (2) shutting down MRI apparatus; (3) shutting down the protective cover of the MRI bore; (4) reporting through an audible or visual signal; and (4) neutralizing the MRI magnet; and any combination thereof.

It is another object of the current invention to disclose the standard of care protocol as defined in any of the above wherein additionally comprising a step of: (a) questioning the subject in case metals were detected to identify their exact location; (b) taking off metals containing objects if possible; (c) re-entering the metal detector gate to check if subject is clear from detectable metals; (d) repeating steps a-c until subject is clear of detachable detectable metals.

It is another object of the current invention to disclose the standard of care protocol as defined in any of the above wherein the metal detector gate is characterized by at least one magnetic field sensor for identifying detectable metals.

It is another object of the current invention to disclose the standard of care protocol as defined in any of the above wherein the metal detector gate is additionally connected to a CPU adapted to process data received from the metal detector gate.

It is another object of the current invention to disclose the standard of care protocol as defined in any of the above wherein the metal detector gate additionally comprising: (a) means for evaluating parameters from a group consisting of (i) type; (ii) distance; (iii) size; (iv) depth; (v) location; and (vi) amount of detectable metals; and any combination thereof; and (b) means for reporting these parameters and any of their combination thereof.

It is another object of the current invention to disclose the standard of care protocol as defined in any of the above wherein the metal detector gate is portable.

It is another object of the current invention to disclose the standard of care protocol as defined in any of the above wherein the metal detector gate is turned on by a remote control.

### BRIEF DESCRIPTION OF THE FIGURES

In the following detailed description of the preferred embodiments, reference is made to the accompanying drawings that form a part hereof, and in which are shown by way of illustration specific embodiments in which the invention may be practiced. It is understood that other embodiments may be utilized and structural changes may be made without departing from the scope of the present invention. The present invention may be practiced according to the claims without some or all of these specific details. For the purpose of clarity, technical material that is known in the technical fields related to the invention has not been described in detail so that the present invention is not unnecessarily obscured. In the accompanying drawing:

FIG. 1 is a schematic flow diagram illustrating the standard of care protocol for scanning patient for detectable metals in order to reduce the occurrence of gossypiboma during an open surgery (100);

FIGS. 2A, 2B and 2C schematically illustrate surgical items adapted to assist in the prevention of gossypiboma (200). FIG. 2A schematically illustrates non-metallic surgical items marked with a metallic indicium (210). FIG. 2B schematically illustrates un-marked metallic surgical items (220). FIG. 2C schematically illustrates metallic surgical items with metallic indicia (230).

FIG. 3 is a schematic flow diagram illustrating the standard of care protocol for scanning a patient for metals in order to reduce the occurrence of gossypiboma during minimally invasive surgery (300);

FIG. 4 is a schematic illustration of an endoscope embedded to a metal detector (400);

FIG. 5 is a schematic flow diagram illustrating the standard of care protocol for scanning a patient for metals to reduce the occurrence of gossypiboma when using a toolbox system in a minimally invasive surgery (500);

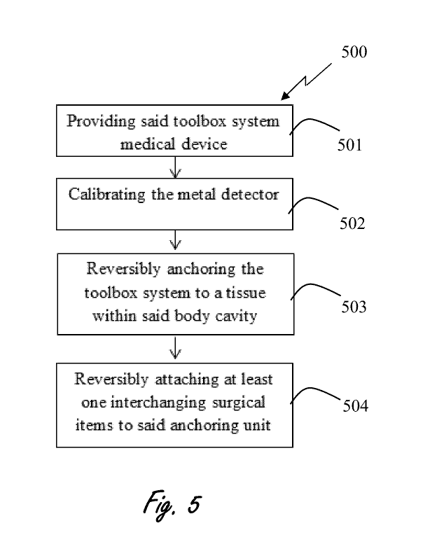

FIG. 6 is a schematic flow diagram illustrating the standard of care protocol for screening a subject for metals prior his entrance to an MRI room using a metal detector embedded to a portable medical device (600); and

FIG. 7 is as a schematic representation of the MRI room (700).

### DETAILED DESCRIPTION OF THE PREFERRED EMBODIMENTS

The present invention provides standard of care protocols for screening subjects for detectable metals in the purpose of increasing safety in the MRI room and to prevent gossypiboma.

The term “portable medical device” refers hereinafter to any portable medical device, such as an otoscope, stethoscope, CPR Masks, goniometers, thermometer, etc. More generally, the term refers to any medical equipment interfacing the outer (Thermometers, stethoscope, reflex hammerthroat illuminator, laryngeal minor, nasal speculum ,tongue depressor, etc.) or inner (endoscopes, sigmoidoscope, proctoscope, rhinoscope, bronchoscope, otoscope, gynoscope, laparoscope, amnioscope, laryngoscope, esophagoscope, bronchoscope and epiduroscope).

The term “gossypiboma” refers hereinafter to a technical term for a surgical complications resulting from foreign materials, such as a surgical sponge, accidentally left inside a patient's body. This term also refers to any other retained surgical tool (RST) in a patient cavity after a surgical procedure. More generally the term refers to disposable items, non-disposable items, metallic tools and non-metallic tools.

The term “surgical items” refers hereinafter to instrument and tools used during surgery and placed inside the interior of a subject. More generally the term refers to any disposable item (sponges, needles, tape etc.) and non-disposable item (graspers, forceps, clamps, occluders, retractors, ribs and other tissue distractors, positioners, stereotactic devices, mechanical cutters scalpels, lancets, drill bits, rasps, trocars, ligasure, harmonic scalpel, dilators and specula, suction tips and tubes, sealing devices, surgical staplers, irrigation and injection needles, tips and tubes, tyndallers, drills, dermatomes etc.).

The term “metal detector” refers hereinafter to a device which responds to metal that may not be readily apparent. More generally the term refers to any type of metal detector such as Very Low Frequency detectors, Pulse Induction detectors, Beat Frequency Oscillation, etc.

The term “magnetic field sensor” refers hereinafter to the metal detector component that first reacts to the presence of metal by emitting a voltage signal (consists of one or more coils).

The term “detectable metals” refers hereinafter to any metal that produces a magnetic field in response to an applied magnetic field such as ferromagnetic (cobalt, iron, nickel, gadolinium, dysprosium, permalloy, awaruite, wairakite, magnetite, etc.) and ferrimagnetic metals.

The term “reporting mechanism” refers hereinafter to at least one way of indication to the user of the metal detector on the presence of detectable metals in or on the subject. Indicators may be audible, visual or sensible.

The term “CPU-central processing unit” refers hereinafter to the hardware within a computer that carries out the instructions of a computer program by performing the basic arithmetical, logical, and input/output operations of the system.

The term “portable medical device” refers hereinafter to any portable medical device, such as an otoscope, stethoscope, CPR Masks, goniometers, thermometer, etc. More generally, the term refers to any medical equipment interfacing the outer (Thermometers, stethoscope, reflex hammerthroat illuminator, laryngeal minor, nasal speculum, tongue depressor, etc.) or inner (endoscopes, sigmoidoscope, proctoscope, rhinoscope, bronchoscope, otoscope, gynoscope, laparoscope, amnioscope, laryngoscope, esophagoscope, bronchoscope and epiduroscope).

The term “pre-surgery detectable metals in a patient” refers hereinafter to any non-removable detectable metal within a subject (pacemaker, implantable cardioverter defibrillator (ICD), neurostimulator, aneurysm clip , metal implant, implanted drug infusion device, foreign metal objects, shrapnel or bullet wounds, permanent cosmetics or tattoos, dentures/teeth with magnetic keepers, other implants that involve magnets, medication patch that contains metal foil, etc.).

The term “audible indicators” refers hereinafter to a representation of sound, typically as an electrical voltage. Audible indicators have frequencies in the audio frequency range of roughly 20 to 20,000 Hz (the limits of human hearing). Audible indicators may be synthesized directly, or may originate at a transducer such as a microphone, musical instrument pickup, phonograph cartridge, or tape head.

The term “signal” refers hereinafter to any indicator that serves for means of communication.

The term “visual indicators” refers hereinafter to a representation of light in the visible light range of about 380 nanometers to about 740 nm. More generally the terms refer to any light within the visible range that will be noticeable by the user of the invention (light, flashing light, flickering light, blinking light, change of spectrum of colors of light etc.).

The term “sensible indicators” refers hereinafter to a physical movement of the medical device embedding the metal detector which is noticeable to the user (shaking, vibrating, quivering, etc.).

The term “signal intensity” refers hereinafter to the electric-field strength of the electromagnetic wave transmitting a signal.

The term “toolbox system” refers hereinafter to a system adapted to change instruments during minimal invasive surgery within the body cavity. It is characterized by at least one anchoring unit adapted to be partially reversibly anchored to an internal wall within a body cavity and at least one interchanging metal comprising surgical item adapted to be reversibly attached to the anchoring unit.

The term “non-metallic surgical items” refers hereinafter to any surgical item that is not made of metal and cannot be detected by a metal detector. More generally the term refers to pads, sponges, tapes, gauzes, dressings, stitches, threads, sutures, tubes, plastic items, rubber items.

The term “metallic surgical items” refers hereinafter to any surgical item made at least partially of metal that can be detected by a metal detector. More generally the term refers to: graspers, forceps, clamps, occluders, retractors, ribs and other tissue distractors, positioners, stereotactic devices, mechanical cutters scalpels, lancets, drill bits, rasps, trocars, ligasure, harmonic scalpel, dilators and specula, suction tips and tubes, sealing devices, surgical staplers, irrigation and injection needles, tips and tubes, tyndallers, drills, dermatomes.

The term “interchanging surgical item” refers hereinafter to any surgical item that can be reversibly attached to the anchoring unit of the toolbox system. More generally, the items can be metal made or non-metal made with a detectable metallic indicia.

The term “interchanging metallic surgical items” refers hereinafter to any surgical item made of metal that can be reversibly attached to the anchoring unit of the toolbox system and cannot be detected by a metal detector. More generally the term refers to graspers, forceps, clamps, occluders, retractors, ribs and other tissue distractors, positioners, stereotactic devices, mechanical cutters scalpels, lancets, drill bits, rasps, trocars, ligasure, harmonic scalpel, dilators and specula, suction tips and tubes, sealing devices, surgical staplers, irrigation and injection needles, tips and tubes, tyndallers, drills, dermatomes.

The term “interchanging non-metallic surgical items” refers hereinafter to any surgical item not made of metal that can be reversibly attached to the anchoring unit of the toolbox system and cannot be detected by a metal detector. sutures, tubes, plastic items, rubber items.

The term “stethoscope” refers hereinafter to an acoustic medical device for auscultation, or listening to the internal sounds of an animal or human body. It operates by the transmission of sound from the chest piece, via air-filled hollow tubes, to the listener's ears. The tubes are also used to carry around the neck when not in use. More generally, the term refers to any kind of stethoscope (acoustic, electronic, recording, fetoscope and Doppler stethoscope).

The term “body cavity” refers hereinafter to any space in an organism that is filled by fluid. The human body consists of the following body cavities: cranial cavity, spinal canal, ventral body cavity, thoracic cavity, abdomen pelvic cavity, abdominal cavity and pelvic cavity.

The term “minimally invasive surgery” refers hereinafter to a procedure that is carried out by entering the body through the skin, a body cavity or an anatomical opening, but with the smallest damage possible. This term include endoscopy, laparoscopy, NOTE (Natural orifice translumenal endoscopic surgery), NOS (Natural Orifice Surgery), etc.

The term “endoscope” refers hereinafter to the apparatus used in a minimal invasive surgery that comprises:(a) at least one rigid or flexible tube; (b) light delivery system; (c) lens system; (d) eyepiece; and (e) at least one additional channel to allow entry of medical instruments or manipulators. More general the terms refers to: endoscope; sigmoidoscope; proctoscope; rhinoscope; bronchoscope; otoscope; gynoscope; laparoscope; amnioscope; laryngoscope; esophagoscope; bronchoscope; epiduroscope.

The term “distal tip of the endoscope” refers hereinafter to the end of the part of the endoscope that in introduced into the body cavity.

The term “toolbox system” refers hereinafter to a system adapted to change instruments during minimal invasive surgery within the body cavity. It is characterized by at least one anchoring unit adapted to be partially reversibly anchored to an internal wall within a body cavity and at least one interchanging metal comprising surgical item adapted to be reversibly attached to the anchoring unit.

The term “interchanging metal comprising surgical item” refers hereinafter to any surgical item that can be reversibly attached to the anchoring unit of the toolbox system. More generally, the items can be metal made or non-metal made with a detectable metallic indicia.

It is according to one embodiment of the invention, wherein a standard of care protocol for using a metal detector is adapted to prevent gossypiboma during open surgery (100). In this protocol the patient is screened for detectable metals in a time resolved manner to assure no surgical items are retained within a patient's body. In order to prevent signaling in response to detectable metal items previously and permanently fixed within the patient a base line threshold signal is determined according to those items (102). Any signal exceeding this threshold is indicative of an undesirable metallic item within the patient.

It is recommended to screen the patient constantly for detectable metal but if not possible another option is to carry out the screening at least in the following time points: before surgery to determine the base line and after that prior the beginning of the surgery to make sure the baseline was correctly established, before closing, after closing, before moving to the recovery room and before discharge of the patient from the recovery room.

The metal detector can be hand-held either embedded or not embedded to a portable medical device (like a stethoscope) or a permanent metal detector embedded within the surgery room. The metal detector can be further connected to a receiver and a display unit.

The signal produced by the reporting mechanism of the metal detector can either be visual, audible or sensible or any of their combinations. The intensity of the signal is characterized as function of at least one parameter or a combination of them. Possible parameters are: type, size, distance, depth, location, etc.

A visual signal will preferably include a light that turns on when metal is detected or another option where green light indicates no metal while red light indicates the presence of a metal.

The audible signal can be a beep or some other alarming sound heard in a response to metal detection.

A sensible signal is applicable only in the case that the metal detector is carried by a personnel member ad can be in the form of a vibration.

This method is useful for detecting metallic surgical items. However, the items most widely forgotten within a patient are rather made of fabric, such as sponges and pads. In order for the standard of care to be applicable to non-metallic surgical items it is used with items marked with detectable metallic indicia. These indicia will enable the detection of these items by the metal detector.

Metallic surgical items can be marked as well with at least one detectable metallic indicium. The indicium will give the surgical tools either a unified signal that will distinguish them from any other metallic entity. In addition it is possible to give each surgical item a specific indicium that will enable differentiating between them by the metal detector. If the surgical item is made from more than one part it is possible to mark each of the parts in case it falls apart.

In yet another embodiment of the invention, wherein a standard of care protocol for using a metal detector is adapted to prevent gossypiboma during minimally invasive surgeries. In this protocol the patient is constantly screened for detectable metals to assure no surgical items are retained within a patient's body.

o achieve the best results the metal detector is embedded within the endoscope whereas the magnetic field sensor is located in the distal tip of the apparatus while the reporting mechanism is connected to one of the external parts of the apparatus.

Like in open surgery it is possible to detect non-metallic items by marking them with a detectable metallic indicium.

In yet another embodiment of the invention, a standard of care protocol is provided for using a metal detector, adapted to prevent gossypiboma during minimally invasive surgeries when using a toolbox system. In this protocol the metal detector is embedded within the anchoring unit of the toolbox system and is adapted to detect metals in the environment close to the anchoring unit, thus enabling the detection of attachment and detachment of the interchanging metal comprising surgical items. This protocol enables to unequivocally determine that no surgical items were left behind in the body cavity.

As in previous embodiments the interchanging metal comprising surgical items include also items not containing metal by adding to them detectable metallic indicia.

The signal created by the metal detector can be visual, audible or sensible or any combination of them.

A visual signal can appear on the surface of the toolbox or on the internal parts of the invasive surgery apparatus and viewed through the lens system or on the external parts of the endoscope. The reporting mechanism can be connected or additionally connected to the entrance door and by that disable the exit if not all surgical items are present; to the wall of the room or the tool tray or to the uniform of personnel or to items carried by them and by that the signal will be available also to staff people not directly carrying out the procedure.

The visual signal will preferably be a light that can either change color, intensity or amount in accordance to the amount of interchanging metal comprising surgical items connected to the anchoring unit. The most simple and straight forward visual signal will be a red light signaling one of the items are missing from the anchoring unit and a green light signaling they are all present.

In yet another embodiment of the invention, wherein a standard of care protocol for using a metal detector is adapted to screen subjects prior their entrance to the MRI room to assure no metals are present when the MRI's magnet is turned on. The metal detector will alert on any metal containing belongings carried by the subject and any other metal comprising items on or within them. This procedure will remind subjects to take off all metal containing items that are reversibly attached to them and in case of items permanently attached to them (pacemakers, stents, etc.) each case should be considered separately by authorized personnel.

The reporting mechanism of the metal detector can alert on a metal containing object in contact within a subject by sending a visual, audible or sensible signal. It can also cause an action that will disable the proceeding of the MRI exam. For instance, it can cause the entrance door to the MRI room to lock, it can shut down physically or mechanically the MRI apparatus or shut down the protective cover of the MRI bore.

The metal detector can be hand-held and embedded within a portable medical device such as a stethoscope. This way, the user of the metal detector does not even need to remember that he has to check the subject for metals. The metal detector will be working and in case of a metal the user or any other staff member will be warned.

The metal detector can be embedded within at least one part of the stethoscope that includes ear tips, tubing and chest piece.

There is also the possibility of a metal detector gate positioned in the entrance to the MRI room, before the patients table or in the entrance to entrance to the MRI bore. Like in the hand-held metal detector the reporting mechanism of the metal detector can be connected to other apparatus.

The metal detector can also be adapted to locate the metal and estimate its size and amount. This will enable to decide whether a patient carrying the metal can get the exam or the metal is a contraindication for it.

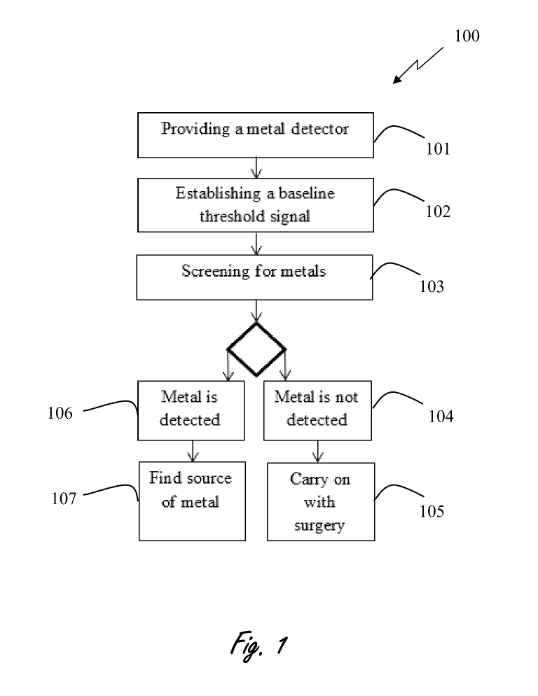

Reference is now made to FIG. 1. A schematic flow diagram illustrating the standard of care protocol for scanning a patient for detectable metals adapted to reduce the occurrence of gossypiboma during an open surgery (100). In the first step a metal detector is provided (101). First, a baseline threshold characteristic of pre-surgery detectable metals in the patient is determined (102). The patient is then screened for newly introduced detectable metals during the different steps of the surgery (103). If metals are detected (104) then the source of metal should be detected (105) and removed in the case that they were accidently left in the patient. If no metal are detected (106) then the surgery can be carried on (107).

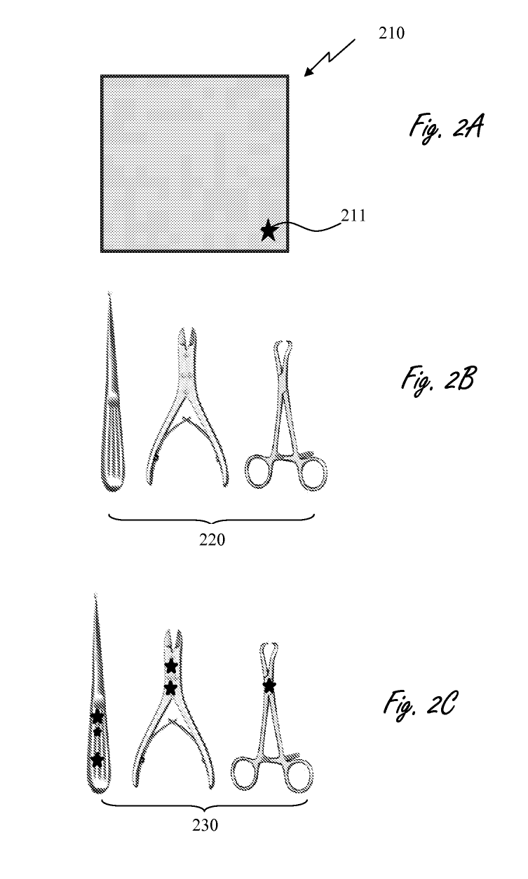

Reference is now made to FIGS. 2A, 2B and 2C. A schematic illustration of surgical items adapted to assist in the prevention of gossypiboma. The surgical items can be detected by a metal detector. Non-metallic surgical items (210) marked with a metallic indicium (211) while metallic surgical items are either not marked (220) or marked (230) with metallic indicia.

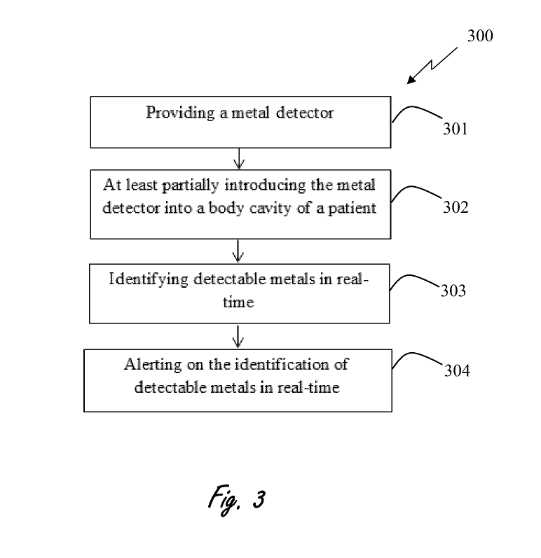

Reference is now made to FIG. 3. A schematic flow diagram illustrating the standard of care protocol for screening a patient for metals to reduce the occurrence of gossypiboma during minimally invasive surgery (300). In the first step the metal detector, embedded to the endoscope is provided (301) and is at least partially introduced to a body cavity of a patient (302). During the procedure, the metal detector identifies metals in the patient's cavity in real-time (303). In the case a metal is detected the signaling mechanism alerts on its presence (304).

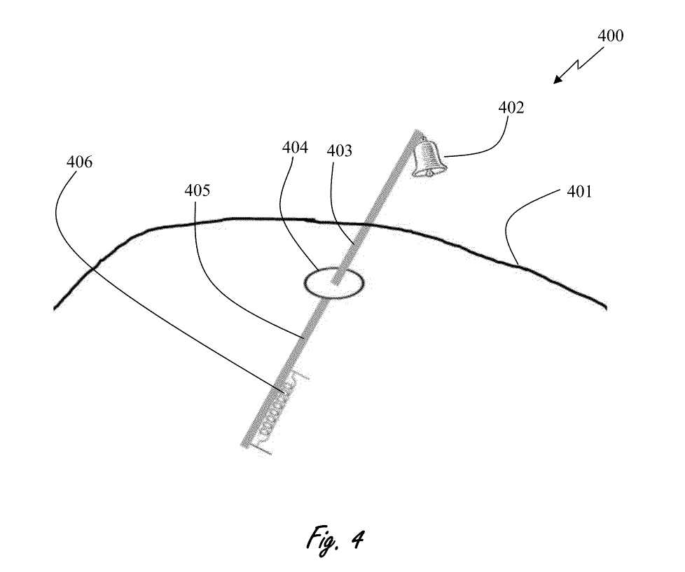

Reference is now made to FIG. 4. A schematic illustration of an endoscope (400) embedded to a metal detector, partially inserted into a cavity (404) of a human body (403) during minimally invasive surgery. The magnetic field sensor (406) is located on the distal part (405) of the endoscope so it could be in close proximity to the site of the surgery. The reporting mechanism (402) is located in the proximal (403) part of the endoscope so it will be visible to the user.

Reference is now made to FIG. 4. A schematic flow diagram illustrating the standard of care protocol for scanning a patient for metals to reduce the occurrence of gossypiboma when using a toolbox system in a minimally invasive surgery (500). In the first step a toolbox system is provided (501) to which a metal detector is embedded. The toolbox is then introduced into the body cavity of a patient (502) and is reversibly anchored to the internal wall of the cavity (503). In the next step interchanging metal comprising surgical items are reversibly attached to the anchoring unit (504). During the procedure, when surgical items are attached and detached the metal detector signal whether any items are missing from the anchoring unit by assessing the amount of metal. Before pulling out the toolbox a checking step is carried out to make sure all items are attached to the anchoring unit.

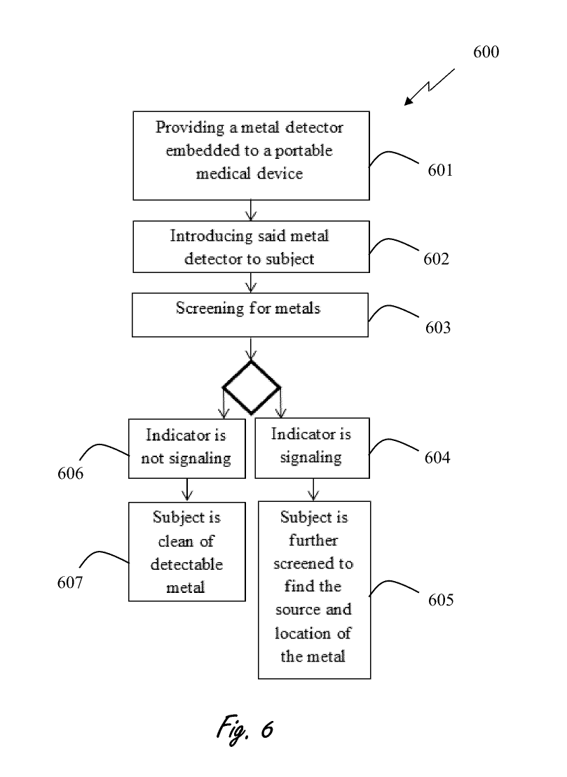

Reference is now made to FIG. 6. A schematic flow diagram illustrating the standard of care protocol for screening a subject for metals prior his entrance to an MRI room using a metal detector embedded to a portable medical device (600). In the first step a metal detector is provided (601). This step might be passively performed since the metal detector is embedded within a portable medical device already carried by staff member responsible for preparing subjects prior their entrance to the MRI room. The metal detector is then introduced to the subject (602). The introducing step, again, can be carried out passively when the staff member approaches the subject. This step can also be actively performed during a physical exam executed by the portable medical device to which the metal detector is embedded. During this direct or indirect interaction of the metal detector with the subject screening for detectable metals is carried out (603). If the metal detector is not signaling for the presence of metals (604) then it can be said that the subject is clean from metals and can safely enter the MRI room (605). If the metal detector signals for the presence of metals (606) then the subject should be further scanned for metals (607).

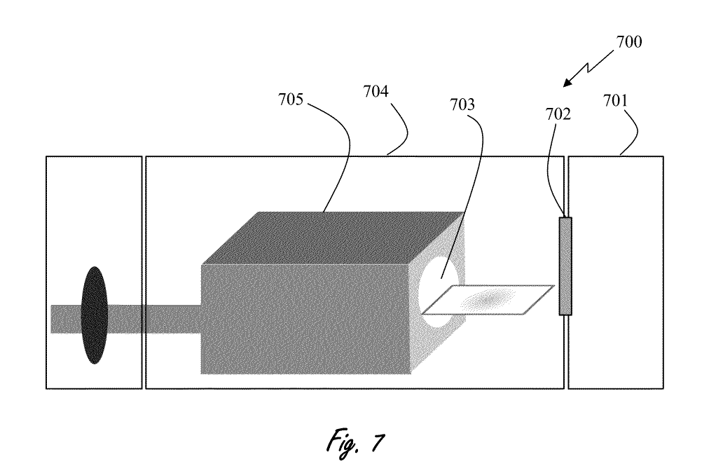

Reference is now made to FIG. 7. A schematic illustration of an MRI room (700). The waiting room (701) is the place where most likely the metal detecting process will take place. If metal is detected above a certain predetermined threshold at least one of the following actions will take place: the entrance door (702) to the MRI room (704) will lock, the cover of the MRI bore (703) will shut down and/or the MRI apparatus (705) will shut down.

1. A standard of care protocol for preventing gossypiboma during surgery, characterized by steps of:
a. selecting one or more surgical items from a group consisting of: non-metallic surgical items marked with detectable metal indicia (210); metallic surgical items (220); metallic surgical items marked with said detectable metal indicia (230) and a combination thereof;
b. providing one or more metal detectors in connection with at least one of said surgical items; and
c. identifying by mean of said at least one metal detector detectable metal items in said patient.

2. The protocol of claim 1, additionally comprising a step of specifying a distinct metal indicium to each said surgical items in a manner selected from a group consisting of: (a) type of detectable metal; (b) geometry; (c) amount of metal and any combination thereof

3. The protocol of claim 1, additionally comprising a step of interconnecting the detectable metal indicia to said surgical item in a way selected from a group consisting of: welding, affixing, embedding, interweaving, coupling, casting, joining, implanting, attaching, linking, screwing, adhering, stamping, forming, bending and any combination thereof.

4. A standard of care protocol for preventing gossypiboma during open surgery (100), comprising steps of:
a. providing a metal detector (101) comprising:
i. at least one magnetic field sensor for identifying detectable metals;
ii. at least one reporting mechanism adapted to signal data concerned with detection of said detectable metals; and,
iii. a CPU for processing said data; and
b. identifying detectable metal items in said patient (103),
wherein said identifying comprises steps of processing said data to establish a baseline threshold signal characteristic of pre-surgery detectable metals in said patient (102), and then identifying signals exceeding said baseline threshold in a time resolved manner during and after said surgery procedure, thereby facilitating the prevention of gossypiboma.

5. The protocol of claim 4, wherein said metal detector is hand-held.

6. The protocol of care of claim 4, additionally a comprising step of embedding said metal detector to an object selected from a group consisting of: (a) within a portable medical device; (b) a surgical implement proximate to the operating room and any combination thereof.

7. The protocol of claim 4, wherein said metal detector additionally comprises:
a. means for evaluating parameters from a group consisting of (a) type; (b) distance; (c) size; (d) depth; (e) location; and (f) amount of detectable metals; and any combination thereof; and
b. means for reporting said parameters and any of their combination thereof by producing a signal.

8. The protocol of claim 4, additionally comprising a step of selecting said signal from a group of indicators consisting of: (a) audible indicators; (b) visual indicators; and (c) sensible indicators; and any combination thereof.

9. The protocol of claim 8, additionally comprising a step of characterizing said signal intensity as a function of at least one parameter from a group consisting of (a) type; (b) distance; (c) size; (d) depth; (e) location; (f) amount of detectable metals; and any combination thereof.

10. A standard of care protocol for preventing gossypiboma during a minimally invasive surgery (300), comprising steps of:
a. providing a metal detector adapted to identify detectable metal items in a body cavity of a patient (301);
b. at least partially introducing said metal detector into a body cavity of said patient (302);
c. identifying said detectable metal items in real-time (303); and
d. alerting on identification of said detectable metal items in real-time (304),
wherein said metal detector is characterized by at least one magnetic field sensor for identifying detectable metals embedded within the distal tip of the endoscope and at least one reporting mechanism adapted to signal detection of said detectable metals embedded within the external parts of the endoscope, thereby facilitating the prevention of gossypiboma.

11. A standard of care protocol for preventing gossypiboma while using a toolbox system for interchanging surgical items within a body cavity during a minimally invasive surgery (500), comprising steps of:
a. providing said toolbox system (501) characterized by:
i. at least one anchoring unit adapted to be at least partially reversibly anchored to an internal wall within said body cavity;
ii. at least one interchanging surgical item adapted to be reversibly attached to said anchoring unit; and
iii. a metal detector embedded within said anchoring unit adapted to detect changes in the amount of said interchanging surgical items attached to it and alert on said changes;
b. calibrating the metal detector according to the number of said interchanging surgical items attached to it (502);
c. reversibly anchoring said toolbox system to a tissue within said body cavity (503); and
d. reversibly attaching at least one interchanging surgical items to said anchoring unit; thereby providing said at least one interchanging metal comprising surgical items within said body cavity (504),
wherein said metal detector is adapted to alert whenever at least one said interchanging surgical items is detached from the anchoring unit, thereby facilitating the prevention of gossypiboma.

12. The protocol according to claim 11, additionally comprising steps of:
a. adapting said a magnetic field sensor to detect changes in the magnetic field in the proximity of said anchoring unit as a result of attaching/detaching said interchanging metal comprising surgical items; and
b. adapting a reporting mechanism to alert upon said changes in the magnetic field.

13. The protocol according to claim 12, additionally a comprising step of detaching said toolbox system from said tissue within said body cavity which activates said metal detector upon said action of detaching.

14. The protocol of claim 11, additionally comprising a step of selecting said interchanging surgical items from a group consisting of: (a) interchanging non-metallic surgical items marked with detectable metal indicia; (b) interchanging metallic surgical items; (c) interchanging metallic surgical items with detectable metal indicia; and any combination thereof.

15. The protocol of claim 12, additionally comprising a step of selecting said reporting mechanism from a group consisting of: (a) visual signal; and (b) audible signal; (c) sensible signal and any combination thereof.

16. The protocol of claim 12, additionally comprising a step of interconnecting said reporting mechanism to an object, and selecting the same from a group consisting of: (a) entrance door; (b) wall; (c) tool tray; (d) personnel uniform; (e) items carried by said personnel; and any combination thereof.

17. A standard of care protocol for increasing MRI safety by screening subjects for detectable metals prior their entrance to the MRI room utilizing a hand-held metal detector (600), comprising steps of:
a. providing said metal detector (601) comprising:
i. at least one magnetic field sensor for identifying detectable metals; and
ii. at least one reporting mechanism adapted to alert upon the detection by said magnetic field sensor;
b. introducing said metal detector to said subject (602);
c. identifying the presence of said detectable metals by said magnetic field sensor; and
d. alerting on the detection of said detectable metals by at least one action from a group consisting of: (1) locking the entrance door to the MRI room (702); (2) shutting down the MRI apparatus (705); (3) shutting down the protective cover of the MRI bore (703); (4) producing an audible or visual alert; and any combination thereof,
wherein said protocol additionally comprising step of embedding said hand-held metal detector within a portable medical device.

18. The protocol of claim 17, further comprising steps of providing said stethoscope with at least one chest piece adapted to be reversibly contacted a subject; at least one ear tip; and a tubing interconnecting said chest piece with said ear tips.

19. The protocol of claim 18, further comprising steps of embedding said magnetic field sensor and said reporting mechanism within a stethoscope's parts selected from a group consisting of (a) chest piece; (b) ear tips; and (c) tubing; and any combination thereof.
